(19) United States
(12) Patent Application Publication
(10) Pub. No.: US 2017/0000449 A1
(43) Pub. Date: Jan. 5, 2017

(54) BREAST IMAGING APPARATUS, METHOD OF BREAST IMAGING APPARATUS, AND STORAGE MEDIUM

(71) Applicant: CANON KABUSHIKI KAISHA, Tokyo (JP)

(72) Inventors: Osamu Tsujii, Kawasaki-shi (JP); Tetsuo Shimada, Hachioji-shi (JP); Nobuhiro Takeuchi, Yokohama-shi (JP); Sakiko Yamaguchi, Tokyo (JP); Takahiro Noguchi, Tokyo (JP); Hitomi Ogasawara, Kawasaki-shi (JP); Chifuyu Inagaki, Tokyo (JP)

(21) Appl. No.: 15/184,377

(22) Filed: Jun. 16, 2016

(30) Foreign Application Priority Data
Jun. 30, 2015 (JP) 2015-131840

(51) Int. Cl. A61B 6/00; A61B 6/03

(52) U.S. Cl. CPC A61B 6/545; A61B 6/502; A61B 6/032; A61B 6/4014; A61B 6/4417

(57) ABSTRACT

A breast imaging apparatus, capable of performing mammogram imaging and CT imaging by a radiation imaging unit that holds a radiation generation unit and a radiation detection unit configured to detect radiation irradiation from the radiation generation unit such that the radiation generation unit and the radiation detection unit face each other, includes: a housing elevating unit configured to vertically move a housing support unit configured to support the radiation imaging unit; and a control unit configured to control the vertical movement of the housing elevating unit based on an imaging type that is one of mammogram imaging and CT imaging.

### BACKGROUND OF THE INVENTION

### Field of the Invention

The present invention relates to a breast imaging apparatus that performs mammography using radiation, a method of controlling the breast imaging apparatus, and a storage medium.

### Description of the Related Art

Japanese Patent Laid-Open No. 2013-538668 discloses, as a breast imaging apparatus, an arrangement having a function of performing CBCT (Cone-Beam CT) imaging of a breast while rotating a radiation generation unit and a radiation detection unit by a rotation unit and a function of performing mammogram imaging while fixing the breast by a fixing unit.

Japanese Patent Laid-Open No. 2013-538668 discloses an arrangement for performing CBCT imaging of a breast of an object in a standing position and an arrangement for performing mammogram imaging. However, an arrangement for moving, based on an imaging type, the radiation detection unit and a support unit that supports the radiation imaging unit is not disclosed. In the arrangement of Japanese Patent Laid-Open No. 2013-538668, an imaging technician needs to adjust the position of a housing support unit so as to form different imaging geometric systems in mammogram imaging and CT imaging.

The present invention provides a breast imaging technique capable of controlling the position of a housing support unit that supports a radiation imaging unit.

### SUMMARY OF THE INVENTION

According to one aspect of the present invention, there is provided a breast imaging apparatus capable of performing mammogram imaging and CT imaging by a radiation imaging unit that holds a radiation generation unit and a radiation detection unit configured to detect radiation irradiation from the radiation generation unit such that the radiation generation unit and the radiation detection unit face each other, comprising: a housing elevating unit configured to vertically move a housing support unit configured to support the radiation imaging unit; and a control unit configured to control the vertical movement of the housing elevating unit based on an imaging type that is one of mammogram imaging and CT imaging.

According to another aspect of the present invention, there is provided a method of controlling a breast imaging apparatus including a radiation imaging unit that holds a radiation generation unit and a radiation detection unit configured to detect radiation irradiation from the radiation generation unit such that the radiation generation unit and the radiation detection unit face each other, and a housing elevating unit configured to vertically move a housing support unit configured to support the radiation imaging unit, and capable of performing mammogram imaging and CT imaging by the radiation imaging unit, comprising: controlling, by a control unit, the vertical movement of the housing elevating unit based on an imaging type that is one of mammogram imaging and CT imaging.

According to still another aspect of the present invention, there is provided a breast imaging apparatus capable of performing mammogram imaging and CT imaging by a radiation imaging unit that holds a radiation generation unit and a radiation detection unit configured to detect radiation irradiation from the radiation generation unit such that the radiation generation unit and the radiation detection unit face each other, comprising: a housing elevating unit configured to vertically move a housing support unit configured to support the radiation imaging unit; and a control unit configured to control the vertical movement of the housing elevating unit.

According to the present invention, it is possible to provide a breast imaging technique capable of controlling, based on an imaging type, the position of a housing support unit that supports a radiation imaging unit. This makes it possible to automatically set different imaging geometric systems in mammogram imaging and CT imaging and raise the throughput of imaging in both mammogram imaging and CT imaging.

Further features of the present invention will become apparent from the following description of exemplary embodiments (with reference to the attached drawings).

### BRIEF DESCRIPTION OF THE DRAWINGS

FIG. 1 is a view showing the outer appearance of a breast imaging apparatus according to an embodiment at the time of mammogram imaging;

FIG. 2 is a view showing the outer appearance of the breast imaging apparatus according to the embodiment at the time of mammogram imaging;

FIG. 3 is a view showing the outer appearance of the breast imaging apparatus according to the embodiment at the time of CBCT imaging;

FIG. 4 is a view showing the outer appearance of the breast imaging according to the embodiment at the time of CBCT imaging;

FIGS. 5A and 5B are views for explaining the imaging operation of the breast imaging apparatus according to the embodiment;

FIG. 6 is a view for explaining processing of the control unit of the breast imaging apparatus according to the embodiment;

FIGS. 7A to 7C are views for explaining the imaging operation of a breast imaging apparatus according to the second embodiment; and

FIG. 8 is a view for explaining processing of the control unit of the breast imaging apparatus according to the second embodiment.

### DESCRIPTION OF THE EMBODIMENTS

Preferred embodiments of the present invention will now be described in detail with reference to the accompanying drawings. Note that the constituent elements described in the embodiments are merely examples. The technical scope of the present invention is determined by the scope of claims and is not limited by the following individual embodiments.

### First Embodiment

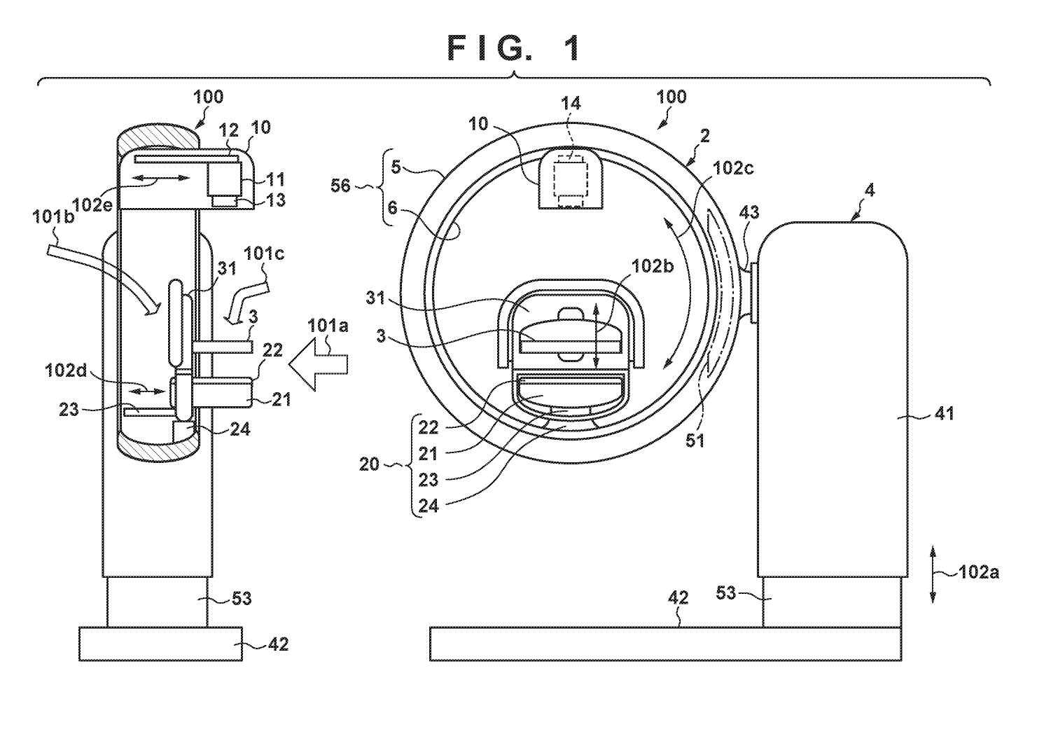

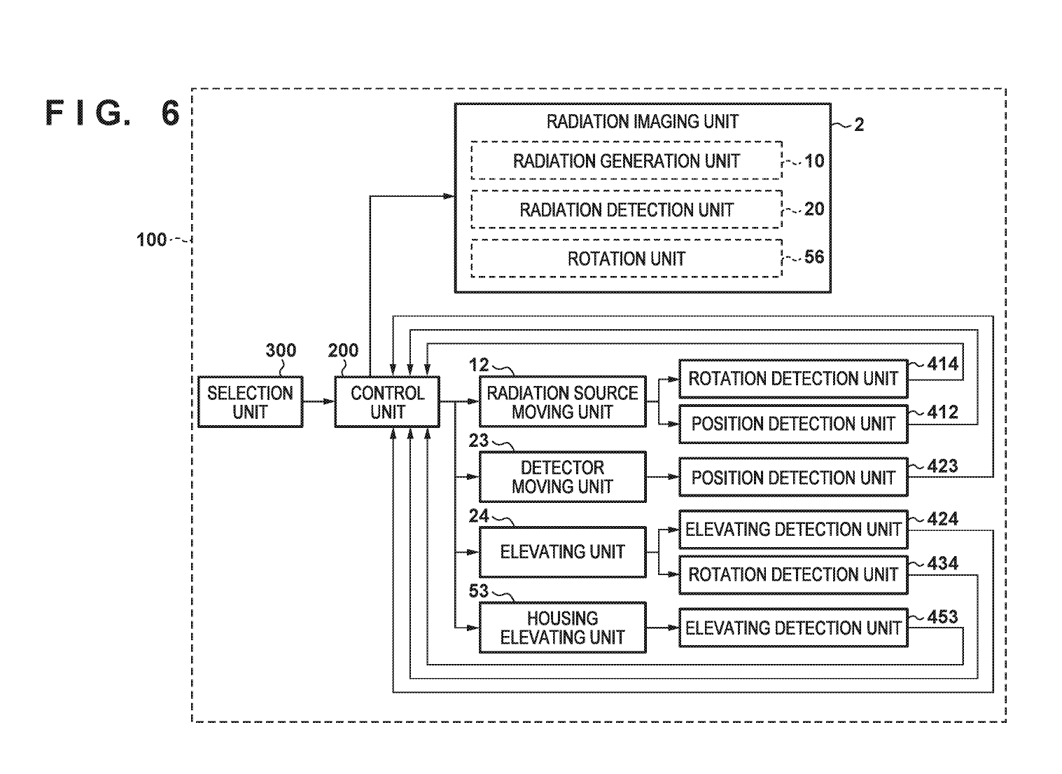

FIG. 1 is a view showing the outer appearance of a breast imaging apparatus 100 according to the first embodiment. The breast imaging apparatus 100 according to the embodiment is a breast imaging apparatus capable of performing mammogram imaging and CT imaging by a radiation imaging unit 2 that holds a radiation generation unit 10 and a radiation detection unit 20 configured to detect radiation irradiation from the radiation generation unit 10 such that they face each other. The breast imaging apparatus includes a housing elevating unit 53 capable of vertically moving a housing support unit 41 configured to support the radiation imaging unit 2, and a control unit 200 (FIG. 6) that controls the vertical movement of the housing elevating unit based on an imaging type that is mammogram imaging or CBCT imaging. The breast imaging apparatus further includes an elevating unit 24 capable of vertically moving the detector of the radiation detection unit 20, and a selection unit 300 (FIG. 6) that selects an imaging type that is mammogram imaging or CBCT imaging. The control unit 200 controls the vertical movement of the housing elevating unit. More specifically, the control unit 200 can control the vertical movements of the housing elevating unit 53 and the elevating unit 24 based on the imaging type. The control unit 200 can control the elevating unit 24 and the housing elevating unit 53 based on the imaging type and automatically set different imaging geometric systems in mammogram imaging and CT imaging. Note that the selection unit 300 has a communication function capable of performing communication (wired communication or wireless communication) with an external apparatus (external system). The selection unit 300 can obtain the imaging type from the external apparatus (external system) by communication. The control unit 200 can control the vertical movement of the housing elevating unit 53 based on the imaging type obtained by not selection of the selection unit 300 but communication of the selection unit 300. The control unit 200 can also control the vertical movement of the elevating unit 24 capable of vertically moving the detector based on the obtained imaging type. That is, the control unit 200 can control the vertical movements of the housing elevating unit 53 and the elevating unit 24 based on the imaging type obtained by communication.

In the first embodiment, an explanation will be made using the breast imaging apparatus 100 capable of performing mammogram imaging and CBCT (Cone-Beam CT) imaging as a radiation imaging apparatus. The radiation imaging unit 2 of the breast imaging apparatus 100 according to this embodiment includes the radiation generation unit 10 that includes a radiation tube 11 (for example, an X-ray tube) serving as a radiation source and generates radiation, the radiation detection unit 20 that includes a radiation detector 21 such as an FPD (Flat Panel Detector) and detects the radiation irradiation from the radiation generation unit 10, and a rotation unit 56 capable of rotating the radiation generation unit 10 and the radiation detection unit 20 in a state in which they face each other.

The rotation unit 56 of the radiation imaging unit 2 includes a ring-shaped rotary frame 6 in which the radiation generation unit 10 and the radiation detection unit 20 are fixed such that they face each other, and a fixed frame 5 that rotatably holds the rotary frame 6 by a rotation sliding member (for example, a bearing). Note that the fixed frame 5 may have an arc shape and rotatably hold part of the rotary frame 6. The rotary frame 6 need not always have a ring shape.

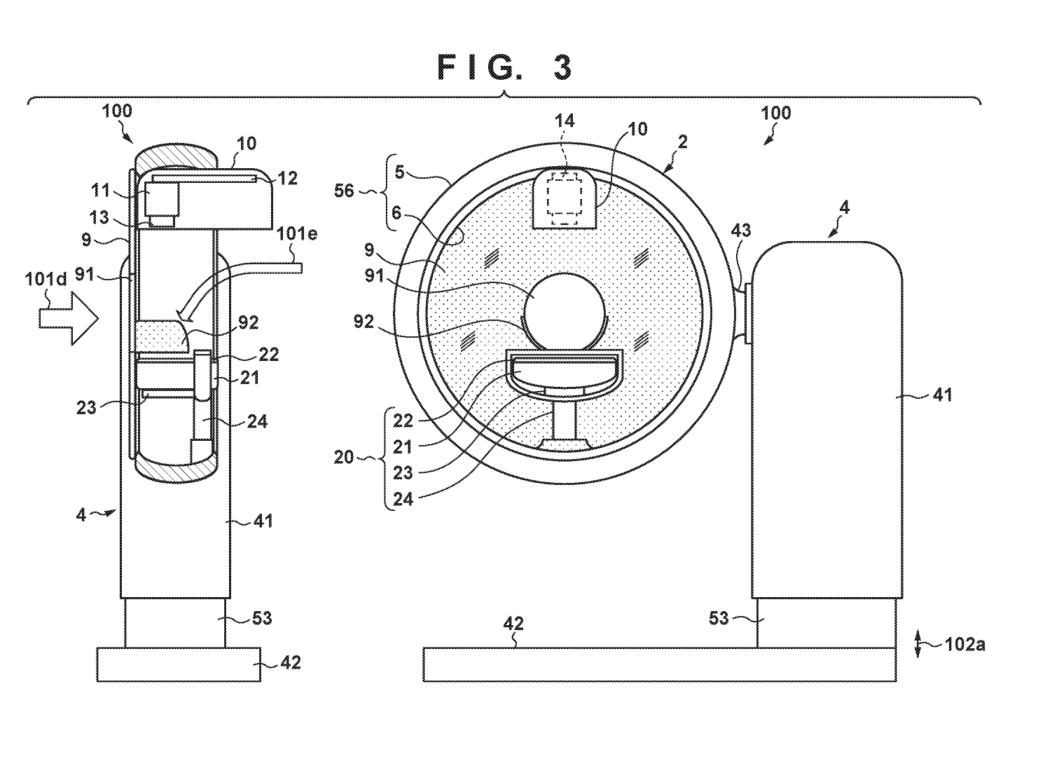

The radiation imaging unit 2 of the breast imaging apparatus 100 is configured to cause a breast that is a body part to be imaged to insert between a pressing plate 3 and the radiation detection unit 20 from the first side (the side of an arrow 101a) of the surface of revolution of the rotary frame 6 for mammogram imaging. The radiation imaging unit 2 of the breast imaging apparatus 100 is also configured to cause a breast that is a body part to be imaged to insert between the radiation generation unit 10 and the radiation detection unit 20 from the second side (an arrow 101d in FIG. 3) opposite to the first side of the surface of revolution of the rotary frame 6 for CT imaging (CBCT imaging in this embodiment).

That is, the radiation imaging unit 2 can implement a mode to perform imaging (mammogram imaging) in a state in which the body part of an object to be imaged is made to insert from the first side in the breast imaging apparatus 100 and sandwiched between the pressing plate 3 and the radiation detection unit 20 and a mode to perform imaging (CBCT imaging) while rotating the radiation generation unit 10 and the radiation detection unit 20 by the rotation unit 56 in a state in which the body part of an object to be imaged is made to insert between the radiation generation unit 10 and the radiation detection unit 20 from the second side opposite to the first side in the breast imaging apparatus 100.

FIG. 1 shows a state in which the breast imaging apparatus 100 captures a CC (Caranio Caudal) view of a mammogram. The rotation position of the rotary frame 6 is decided such that the radiation tube 11, the pressing plate 3, and the radiation detector 21 (radiation detection unit 20) are arranged in the vertical direction. A pressing plate support unit 31 supports the pressing plate 3, and can move the pressing plate 3 in a predetermined direction 102b (for example, a direction in which the pressing plate support unit 31 mounted on the rotary frame 6 moves upward to the rotation center of the rotary frame 6 or a direction in which the pressing plate support unit 31 moves downward to the rotary frame 6). The pressing plate support unit 31 is placed to be removable from the rotary frame 6. Note that the pressing plate support unit 31 may be placed to be removable from a constituent element integrated with the rotary frame 6, for example, the radiation detection unit 20, a detector moving unit 23, or an elevating unit 24. An imaging technician can remove the pressing plate support unit 31 together with the pressing plate 3. The imaging technician can adjust the distance between the pressing plate 3 and the radiation detection unit 20 by moving the pressing plate 3 by the pressing plate support unit 31. The breast of the object can be pressed by moving the pressing plate 3. In mammogram imaging, the breast arranged between the pressing plate 3 and the radiation detection unit 20 is pressed between the pressing plate 3 and the radiation detection unit 20 and undergoes radiation imaging.

The fixed frame 5 of the radiation imaging unit 2 is supported by a housing support unit 41 of a housing unit 4 via a fixed shaft 43. The housing support unit 41 functions as a support unit that supports the radiation imaging unit 2. The housing support unit 41 is configured to be vertically moved by the housing elevating unit 53 with respect to a housing fixing unit 42. The radiation imaging unit 2 is thus supported to be movable in the vertical direction (arrow 102a) with respect to the housing fixing unit 42. When the selection unit 300 (FIG. 6) that selects an imaging type selects an imaging type, the control unit 200 (FIG. 6) switches the imaging type based on the selected imaging type. For example, when changing the imaging type from mammogram imaging to CT imaging (CBCT imaging) or changing the imaging type from CT imaging (CBCT imaging) to mammogram imaging, the control unit 200 controls the vertical movements of the housing elevating unit and the elevating unit based on the imaging type.

For example, the control unit 200 can move the elevating unit 24 based on the imaging type so as to change the distance between the radiation generation unit and the radiation detection unit. In addition, the control unit 200 moves the housing elevating unit 53 based on the imaging type so as to change the position of the housing support unit 41 in the vertical direction. For example, the control unit 200 can obtain a distance based on the difference between SOD (SODmam) in mammogram imaging and SOD (SODct) in CT imaging (CBCT imaging) as the moving distance of the housing elevating unit 53, and move the housing elevating unit 53 based on the obtained distance. Here, SOD (Source to Object Distance) is the distance between the radiation generation unit and the body part of an object to be imaged. In the following description, SOD in mammogram imaging will be referred to as SODmam (first distance), and SOD in CT imaging (CBCT imaging) will be referred to as SODct (second distance).

Note that in the following example, the contents of the control of the control unit 200 using SOD will be described. However, the gist of the present invention is not limited to this. The control unit 200 can also perform the same control using SID (Source to Image Distance) representing the distance between the radiation detector 21 and the radiation generation unit 10. More specifically, SID in mammogram imaging will be referred to as SIDmam, and SID in CT imaging (CBCT imaging) will be referred to as SIDct. The control unit 200 can also obtain a distance based on the difference between SIDmam and SIDct and move the housing elevating unit 53 based on the obtained distance.

When changing the imaging type between mammogram imaging and CT imaging in association with a change in the imaging type selection by the selection unit 300, the control unit 200 obtains a distance based on the difference between the first distance (SODmam) between the radiation generation unit and the body part of the object to be imaged in mammogram imaging and the second distance (SODct) between the radiation generation unit and the body part of the object to be imaged in CT imaging. The control unit 200 then moves the housing elevating unit 53 based on the obtained distance.

In addition, the control unit 200 can control the positions of the radiation tube 11 of the radiation generation unit 10 and the radiation detector 21 of the radiation detection unit 20 based on the selected imaging type. By the control of the control unit 200, for example, even when performing mammogram imaging and CT imaging (CBCT imaging) for the same object or identical objects of different heights, the apparatus can be set in accordance with the position of the body part of the object to be imaged. Detailed control of the control unit 200 will be described later with reference to the block diagram of FIG. 6.

A rotation motor 51 is attached to the distal end of the fixed shaft 43 that connects the housing unit 4 and the radiation imaging unit 2. The rotary frame 6 is rotatably connected to the rotation motor 51 via a bearing. The fixed frame 5 is stationarily connected to the fixed shaft 43. The rotary frame 6 is arranged inside the fixed frame 5. The bearing is arranged in the gap between the fixed frame 5 and the rotary frame 6. By driving the rotation motor 51, the rotary frame 6 can be rotated by 360° or more in a direction indicated by an arrow 102c with respect to the fixed frame 5.

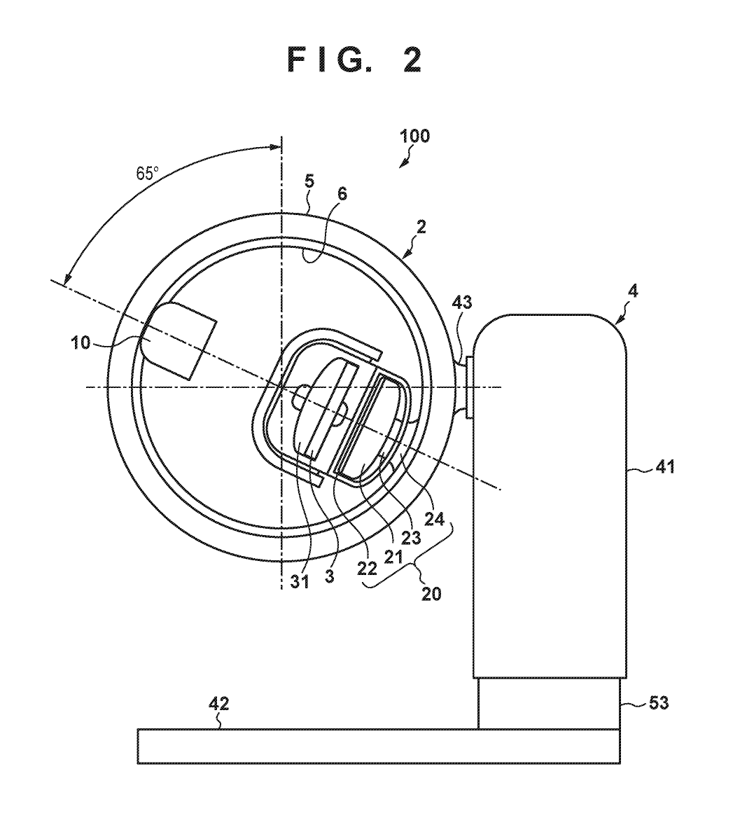

When capturing a CC view of a mammogram, the radiation tube 11, the radiation detector 21, and the pressing plate 3 are arranged in the vertical direction, as shown in FIG. 1. On the other hand, when capturing an MLO (Mediolateral Oblique) view of a mammogram by the breast imaging apparatus 100, the rotary frame 6 is rotated by a predetermined angle (for example, about) 65° from the state shown in FIG. 1 and stopped, as shown in FIG. 2. Note that the stop state of the rotary frame 6 may be maintained by servo control or a brake. The breast that is the body part to be imaged is pressed between the radiation detector 21 and the pressing plate 3 and undergoes radiation imaging. By capturing such an MLO view, imaging of an armpit can be performed.

Referring back to FIG. 1, in mammogram imaging, the imaging technician can access the breast of the object via the hollow portion of the rotary frame 6, as indicated by an arrow 101b, arrange the breast between the pressing plate 3 and the radiation detector 21 of the breast imaging apparatus 100, and adjust the pressing. On the first side that is the breast insertion side upon mammogram imaging, the radiation tube 11, the radiation detector 21, and the pressing plate 3 are fixed such that they project in a first direction with respect to the surface of revolution of the rotary frame 6. For this reason, the imaging technician can also access the breast of the object from a side (between the surface of revolution and the object) of the breast imaging apparatus 100, as indicated by an arrow 101c, and adjust the pressing. The arrangement of the breast imaging apparatus 100 at the time of mammogram imaging has been described above.

CBCT imaging by the breast imaging apparatus 100 will be described next. The radiation generation unit 10 includes a radiation source moving unit 12 capable of moving the radiation source in a direction along the rotation axis of the rotary frame 6. The radiation source moving unit 12 is configured to rotate the radiation source about a rotation axis in a direction intersecting the rotation axis of the rotary frame 6. The control unit 200 can control the rotation of the radiation source by the radiation source moving unit 12 based on the imaging type. Based on the control of the control unit 200, the radiation source moving unit 12 rotates the radiation tube 11 about a rotation axis 14, and moves and arranges the radiation tube 11 in the rotation axis direction (arrow 102e) of the rotary frame 6 to perform mammogram imaging or CT imaging. The radiation source moving unit 12 includes, for example, a rail on which the radiation tube 11 slides, and the imaging technician can manually move the radiation tube 11. Alternatively, the radiation tube 11 may be moved in the direction of the arrow 102e by the driving force of a linear motor or the like. The control unit 200 controls the radiation source moving unit 12 based on the selected imaging type to control the rotation and the position (the position in the translation direction) of the radiation tube 11.

The radiation detection unit 20 includes the detector moving unit 23 capable of moving the radiation detector in a direction along the rotation axis of the rotary frame. The control unit 200 can control the movements of the radiation source moving unit and the detector moving unit based on the imaging type. Based on the control of the control unit 200, the detector moving unit 23 moves and arranges the radiation detector 21 in the rotation axis direction (arrow 102d) of the rotary frame 6 to perform mammogram imaging or CT imaging. The detector moving unit 23 includes a rail on which the radiation detector 21 slides, and the imaging technician can move the radiation detector 21 in the direction of the arrow 102d. Alternatively, the radiation detector 21 may be moved in the direction of the arrow 102d by the driving force of a linear motor or the like. The control unit 200 can control the detector moving unit 23 based on the selected imaging type to control the position of the radiation detector 21 in the translation direction.

The radiation detection unit 20 also includes the elevating unit 24 that moves the radiation detector 21 in the rotation center direction (arrow 102b) of the rotary frame 6 to perform mammogram imaging or CT imaging based on the selected imaging type. The elevating unit 24 is configured to rotate the radiation detector of the radiation detection unit 20 about a rotation axis in a direction intersecting the rotation axis of the rotary frame 6. The control unit 200 can change the direction of the radiation detector 21 of the radiation detection unit 20 by controlling the rotation of the elevating unit 24 based on the imaging type. The direction of the radiation detector 21 can be rotated by rotating the elevating unit 24. The control unit 200 can control the elevating unit 24 based on the selected imaging type to control the rotation and the position (vertical moving position) in the vertical direction of the radiation detector 21.

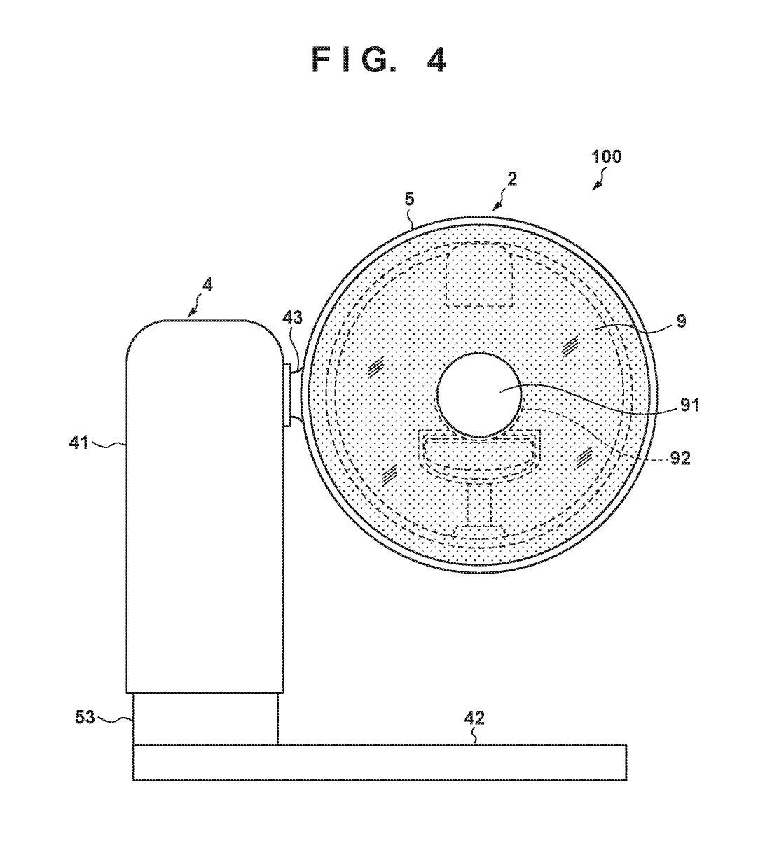

FIG. 3 shows a state in which the breast imaging apparatus 100 according to this embodiment performs CBCT imaging of a breast of an object. FIG. 4 is a view showing the outer appearance of the breast imaging apparatus 100 from the direction of the arrow 101d that is the insertion direction of the breast of the object. At the time of CBCT imaging, the breast is inserted from the second side opposite to the breast insertion side (first side) at the time of mammogram imaging (arrow 101d). In addition, the radiation tube 11 and the radiation detector 21 are moved to the second side opposite to the first side and arranged by the radiation source moving unit 12 and the detector moving unit 23. The radiation source moving unit 12 and the detector moving unit 23 may be configured to move the radiation tube 11 and the radiation detector 21 manually or by motor driving or the like based on the control of the control unit 200. The radiation source moving unit 12 and the detector moving unit 23 can arrange the radiation tube 11 and the radiation detector 21 at positions where mammogram imaging can be executed for a breast inserted from the first side, and CBCT imaging can be executed for a breast inserted from the second side.

The pressing plate support unit 31 and the pressing plate 3 are removable from the rotary frame 6, that is, the radiation imaging unit 2. If the pressing plate support unit 31 and the pressing plate 3 are kept placed on the rotary frame 6, they hinder the imaging technician from accessing the breast of the object when performing CBCT imaging. Hence, at the time of CBCT imaging, the pressing plate support unit 31 is removed from the rotary frame 6 together with the pressing plate 3, as shown in FIGS. 3 and 4.

The elevating unit 24 of the radiation detection unit 20 moves the radiation detector 21 toward the rotation center of the rotary frame 6, thereby changing the distance between the radiation detector 21 and the radiation generation unit 10 (radiation tube 11). The radiation tube 11 and the radiation detector 21 are thus arranged in a positional relationship appropriate for CBCT imaging or mammogram imaging. On the second side of the radiation imaging unit 2 (fixed frame 5), a front cover 9 that separates the object from the radiation imaging unit is configured to be removable from the breast imaging apparatus. The front cover 9 has a function of preventing the object from interfering with the radiation detector 21 and the like when the rotary frame 6 rotates in CBCT imaging. This can ensure safety for the object when performing imaging. The front cover 9 that separates the object from the radiation imaging unit is circular, and is placed to be removable from the circular fixed frame 5. Note that the front cover 9 need only be fixed to a member immovable with respect to the rotation of the rotary frame 6, and may be placed on, for example, the fixed shaft 43.

The front cover 9 that separates the object from the radiation imaging unit is provided with an opening 91 to insert the body part of the object to be imaged. More specifically, the circular opening 91 used to make the breast of the object insert is provided at the center of the front cover 9. The front cover 9 includes, around the opening 91, a breast support 92 used to support the breast that has inserted from the opening 91. Note that in this embodiment, the breast support 92 is fixed to the front cover 9. However, the present invention is not limited to this. For example, the breast support 92 may be fixed to the fixed frame 5 via a support member.

During CBCT imaging, radiation images are captured while rotating the rotary frame 6 with respect to the fixed frame 5, and a reconstruction unit (not shown) obtains a 3D reconstructed image. The front cover 9 fixed to the fixed frame 5 separates the object (not shown) from the radiation generation unit 10 and the radiation detection unit 20 which rotate during CBCT imaging. The breast of the object is held on the breast support 92 and therefore fixed during CBCT imaging.

Note that in the above-described example, the breast support 92 is connected along the periphery of the opening of the front cover 9. However, the present invention is not limited to this. For example, the breast support 92 need only be held immovably with respect to the rotation of the rotary frame 6 during CBCT imaging, and may be connected to, for example, the fixed frame 5. However, when the breast support 92 is connected to the front cover 9, a support member used to connect the fixed frame 5 and the breast support 92 is unnecessary. Hence, the imaging technician can easily access the breast of the object from the direction of an arrow 101e. In FIGS. 3 and 4, the front cover 9 has a removable form. However, the front cover 9 may have an opening/closing structure without hindrance to mammogram imaging.

The imaging technician accesses the breast of the object that has inserted from the first side of the fixed frame 5 and the rotary frame 6 via the opening 91 of the front cover 9, as indicated by the arrow 101e, and places the breast of the object on the breast support 92. Note that the front cover 9 can be configured to be transparent on both the first side and the second side. The front cover 9 can also be configured to be opaque on the object side (the side of the arrow 101d: second side) and transparent on the imaging technician side (the side of the arrow 101a in FIG. 1: first side). The front cover 9 formed to be opaque on the object side can prevent the object from becoming frightened by viewing the movement of the radiation generation unit 10 or the radiation detection unit 20 through the front cover 9. In addition, the front cover 9 formed to be transparent on the imaging technician side allows the imaging technician to visually confirm the state of the object and easily access the breast of the object.

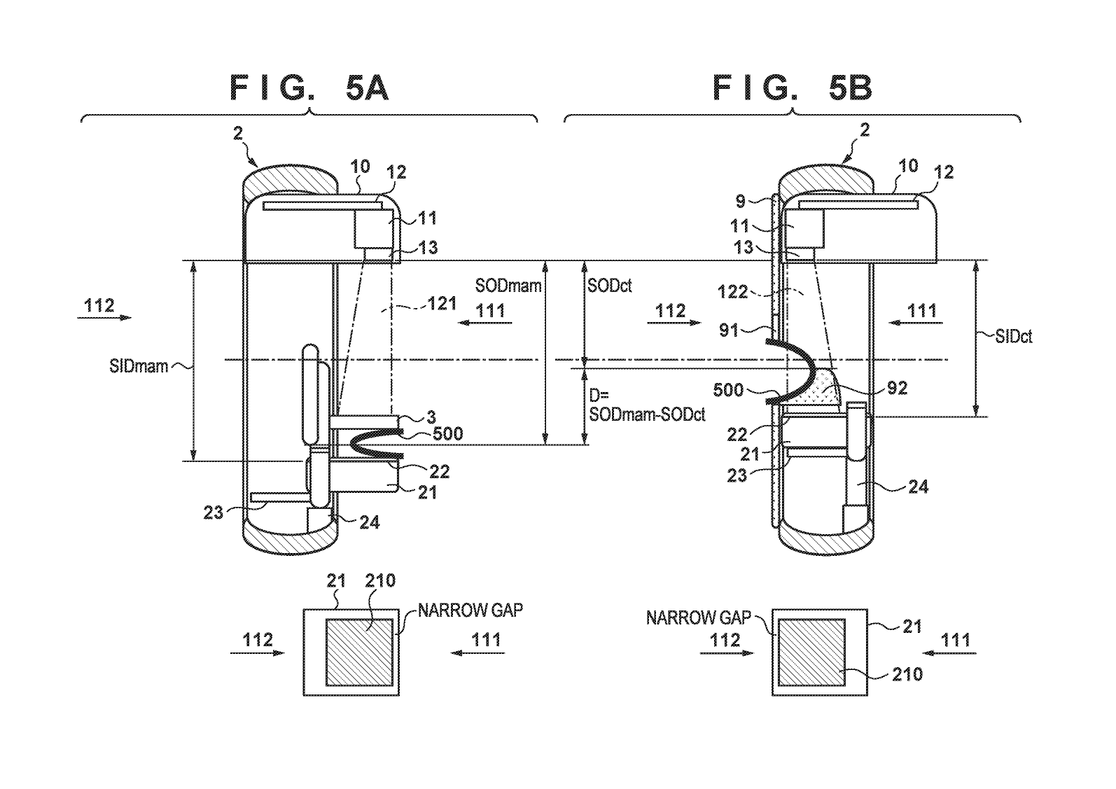

FIGS. 5A and 5B are views showing the mammogram imaging state and the CBCT imaging state of the breast imaging apparatus 100 according to this embodiment. As shown in FIGS. 5A and 5B, the access surface with respect to the rotary frame 6 for the object is reversed between mammogram imaging and CBCT imaging. The side of the access surface for the object at the time of mammogram imaging is defined as a first side 111, and the side of the access surface for the object in CBCT imaging is defined as a second side 112. When selection unit 300 selects an imaging type, the control unit 200 controls the radiation source moving unit 12 based on the selected imaging type to control the rotation and the position (the position in the translation direction) of the radiation tube 11. Additionally, based on the selected imaging type, the control unit 200 controls the detector moving unit 23 to control the position of the radiation detector 21 in the translation direction, and controls the elevating unit 24 to control the rotation and the position (vertical moving position) in the vertical direction of the radiation detector 21.

When switching the imaging type, the control unit 200 controls the movement of the housing elevating unit 53 in accordance with the imaging type. More specifically, the control unit 200 controls the housing elevating unit 53 to move the housing support unit 41 by a distance D based on the difference between SOD (SODmam) in mammogram imaging and SOD (SODct) in CBCT imaging, thereby controlling the position (vertical moving position) in the vertical direction of the radiation imaging unit 2 connected to the housing support unit 41.

FIG. 5A is a side view in mammogram imaging. The object stands on the first side 111. In FIG. 5A, the rotary frame 6 is located at a position corresponding to CC imaging. In MLO imaging, the rotary frame 6 is rotated by about 65° (see FIG. 2). In the radiation generation unit 10, the radiation tube 11 is connected to the rotary frame 6 via the radiation source moving unit 12. The radiation detector 21, the pressing plate support unit 31, the pressing plate 3, and the like are connected to the rotary frame 6 via the elevating unit 24.

By the radiation generation unit 10 and the radiation detection unit 20 including these components, the radiation imaging unit 2 provides different imaging geometric systems in mammogram imaging and CT imaging. Different imaging geometric systems (SID (Source to Image Distance) and SOD (Source to Object Distance)) can thus be provided in mammogram imaging and CBCT imaging.

In addition, since the radiation tube 11 and the radiation detector 21 project to the first side 111 with respect to the surface of revolution of the rotary frame 6 and the fixed frame 5, the imaging technician can access a breast 500 of the object from a side in mammogram imaging (the arrow 101c in FIG. 1). In addition, when the front cover 9 placed on the second side 112 is removed, the imaging technician can access the breast 500 of the object from the second side 112 via the hollow portion of the rotary frame 6 (the arrow 101b in FIG. 1).

Additionally, a radiation collimator 13 is placed in front of the radiation tube 11, and a grid 22 for scattered ray reduction is arranged in front of the radiation detector 21. Since the imaging geometric system changes between mammogram imaging and CBCT imaging, the radiation collimator 13 changes the collimator shape in accordance with mammogram imaging or CBCT imaging. Note that deformation of the opening shape of the radiation collimator 13 can be implemented by an arrangement that deforms the opening shape in accordance with a switching operation of the imaging technician or by exchanging the radiation collimator 13. In addition, the stripe direction, stripe frequency, and grid ratio of the grid 22 are also set in accordance with mammogram imaging or CBCT imaging. For example, the imaging technician exchanges the grid between mammogram imaging and CBCT imaging, thereby coping with each imaging mode.

FIG. 5B is a side view in CBCT imaging. In the breast imaging apparatus 100 according to this embodiment, the control unit 200 can change the form of mammogram imaging shown in FIG. 5A to the form of CBCT imaging shown in FIG. 5B by controlling the radiation source moving unit 12, the detector moving unit 23, the elevating unit 24, and the housing elevating unit 53. That is, the imaging technician removes the pressing plate 3 (pressing plate support unit 31) from the breast imaging apparatus 100 shown in FIG. 5A. The control unit 200 controls the radiation source moving unit 12 to rotate the radiation tube 11 and move it to the second side 112, and controls the detector moving unit 23 to move the radiation detector 21 to the second side 112. The control unit 200 also controls the elevating unit 24 to move the radiation detector 21 upward in FIG. 5A. The control unit 200 also controls the housing elevating unit 53 based on the imaging type to move the housing support unit 41 by the distance D based on the difference between SODmam in mammogram imaging and SODct in CBCT imaging, thereby controlling the position (vertical moving position) in the vertical direction of the radiation imaging unit 2 connected to the housing support unit 41. When changing the imaging type from mammogram imaging to CT imaging, the control unit 200 moves the housing support unit 41 downward according to the distance based on the difference. When the housing support unit 41 moves downward, the radiation imaging unit 2 connected to the housing support unit 41 moves downward. The control unit 200 moves the housing support unit 41 such that the distance based on the difference becomes zero.

Note that an arrangement for moving the radiation detector 21 in the horizontal direction may be implemented by, for example, rotating the radiation detector 21 about the rotation axis of the elevating unit 24 as the rotation center. However, if the radiation detector 21 that has rotated interferes with the rotary frame 6, the radiation detector 21 is configured to be rotatable, for example, after it is raised by the elevating unit 24 to the vicinity of the center of the rotary frame 6.

When changing the form of CBCT imaging shown in FIG. 5B to the form of mammogram imaging shown in FIG. 5A, for example, the control unit 200 rotates the radiation tube 11, moves the radiation tube 11 and the radiation detector 21 to the first side 111, and moves the radiation detector 21 downward in FIG. 5B by the elevating unit 24. The control unit 200 also controls the housing elevating unit 53 to move the housing support unit 41 by the distance D based on the difference between SODmam in mammogram imaging and SODct in CBCT imaging, thereby controlling the position (vertical moving position) in the vertical direction of the radiation imaging unit 2 connected to the housing support unit 41. When changing the imaging type from CT imaging to mammogram imaging, the control unit 200 moves the housing support unit 41 upward according to the distance based on the difference. When the housing support unit 41 moves upward, the radiation imaging unit 2 connected to the housing support unit 41 moves upward. The imaging technician mounts the pressing plate 3 (pressing plate support unit 31) on the radiation detection unit 20.

In CBCT imaging, the object stands on the second side 112. The breast 500 of the object can be aligned with the opening 91 by vertically moving the housing support unit 41. For example, the radiation imaging unit 2 is moved downward by a distance indicated by an arrow 131, thereby aligning the breast 500 of the object with the opening 91. As described above, the pressing plate support unit 31 and the pressing plate 3 have removable structures and are removed in CBCT imaging. In addition, since the access surface for the object changes between mammogram imaging and CBCT imaging, the radiation tube 11 is placed so as to rotate by 180° when moving from the first side 111 to the second side 112. A radiation beam is formed to reduce the blind area (area that is not imaged) of the chest wall portion of the object small, as indicated by radiation beam shapes 121 and 122 in FIG. 5A and 5B. Since the radiation beam is asymmetrical, the radiation tube 11 needs to be rotated. Note that a radiation beam suitable for each imaging may be formed by the radiation collimator 13 without rotating the radiation tube 11. That is, the radiation generation unit 10 can rotate the radiation shape (the radial shape of radiation) from the radiation tube 11 serving as a radiation source by 180° about the radial direction from the rotation center of the rotary frame 6 between mammogram imaging and CBCT imaging.

The detector moving unit 23 can mount the radiation detector 21 in a state in which the radiation detector 21 is rotated by 180° about the radial direction from the rotation center of the rotary frame 6 between mammogram imaging and CBCT imaging. The elevating unit 24 is configured to rotate the radiation detector 21 of the radiation detection unit 20 about a rotation axis in a direction intersecting the rotation axis of the rotary frame 6. The radiation detector 21 can also be rotated by 180° by rotating the elevating unit 24 and arranged in this state. The radiation detector 21 of the radiation detection unit 20 has a narrow gap indicating that the width of one side of the detection area is narrower than the widths of the remaining sides (FIG. 5C). The control unit 200 controls the rotation of the elevating unit 24 based on the imaging type such that the narrow gap of the radiation detector 21 is directed to the object.

The narrow gap of the radiation detector 21 is directed to the object because the access surface for the object changes between mammogram imaging and CBCT imaging. For example, the radiation detector 21 for mammography has a narrow gap (the distance from the outer edge of the sensor to a detection area 210 is 5 mm or less) along only one side of the detection area 210 to reduce the blind area of the chest wall portion, as shown in FIGS. 5A and 5B. For this reason, the radiation detector 21 can be moved and placed so as to rotate by 180° such that the narrow gap side is directed to the object, as shown in FIGS. 5A and 5B. Note that in the arrangement that rotates the radiation detector 21 about the rotation axis of the elevating unit 24 as the rotation center to move the radiation detector 21, the narrow gap side is directed to the object by the rotation.

FIG. 6 is a block diagram for explaining processing of the control unit 200 of the breast imaging apparatus 100. The selection unit 300 can select an imaging type that is mammogram imaging or CT imaging based on an input by the imaging technician via a user interface (display unit) (not shown). The imaging type selected by the selection unit 300 is input to the control unit 200. The control unit 200 can control the operations of the radiation generation unit 10, the radiation detection unit 20, and the rotation unit 56 included in the radiation imaging unit 2 based on the selected imaging type. For example, based on the selected imaging type, the control unit 200 can perform switching control of imaging from mammogram imaging to CT imaging (CBCT imaging) or switching control (imaging control) of imaging from CT imaging (CBCT imaging) to mammogram imaging.

When executing imaging control, based on the selected imaging type, the control unit 200 controls the elevating unit 24 and provides different imaging geometric systems (SID (Source to Image Distance) and SOD(Source to Object Distance)) in mammogram imaging and CT imaging. The rotation of the radiation tube 11 by the radiation source moving unit 12 is detected by a rotation detection unit 414. The detection result of the rotation detection unit 414 is input to the control unit 200. Based on the detection result of the rotation detection unit 414, the control unit 200 performs feedback control of the rotation of the radiation tube 11 and rotates the radiation tube 11 by only a predetermined rotation angle (for example, 180°). The translation of the radiation tube 11 by the radiation source moving unit 12 is detected by a position detection unit 412. The detection result of the position detection unit 412 is input to the control unit 200. Based on the detection result of the position detection unit 412, the control unit 200 performs feedback control of the translation of the radiation tube 11 and moves the radiation tube 11 to a predetermined position.

The translation of the radiation detector 21 by the detector moving unit 23 is detected by a position detection unit 423. The detection result of the position detection unit 423 is input to the control unit 200. Based on the detection result of the position detection unit 423, the control unit 200 performs feedback control of the translation of the radiation detector 21 and moves the radiation detector 21 to a predetermined position.

The rotation of the radiation detector 21 about the rotation axis of the elevating unit 24 is detected by a rotation detection unit 434. The detection result of the rotation detection unit 434 is input to the control unit 200. Based on the detection result of the rotation detection unit 434, the control unit 200 performs feedback control of the rotation of the radiation detector 21 and rotates the radiation detector 21 by only a predetermined rotation angle (for example, 180°). The vertical movement of the radiation detector 21 by the elevating unit 24 is detected by an elevating detection unit 424. The detection result of the elevating detection unit 424 is input to the control unit 200. Based on the detection result of the elevating detection unit 424, the control unit 200 performs feedback control of the vertical movement of the radiation detector 21 and vertically moves the radiation detector 21 to a predetermined position.

The vertical movement of the housing support unit 41 by the housing elevating unit 53 is detected by an elevating detection unit 453. The detection result of the elevating detection unit 453 is input to the control unit 200. Based on the detection result of the elevating detection unit 453, the control unit 200 performs feedback control of the vertical movement of the housing support unit 41 and vertically moves the housing support unit 41 to a predetermined position. When the vertical movement of the housing support unit 41 is controlled by the control unit 200, the radiation imaging unit 2 connected to the housing support unit 41 also vertically moves. That is, the control unit 200 controls the vertical movements of the housing support unit 41 and the radiation imaging unit 2 connected to the housing support unit 41 based on the detection result of the elevating detection unit 453. As described above, the control unit 200 can provide imaging geometric systems (SID (Source to Image Distance) and SOD (Source to Object Distance)) corresponding to mammogram imaging and CBCT imaging by feedback control.

When mammogram imaging is selected, the control unit 200 controls the radiation source moving unit 12, the detector moving unit 23, the elevating unit 24, and the housing elevating unit 53 to control the rotation and translation of the radiation tube 11, the rotation and translation of the radiation detector 21, and the movement of the housing support unit 41 (radiation imaging unit 2) in the vertical direction so as to form the imaging geometric system shown in FIG. 5A.

When CT imaging (CBCT imaging) is selected from the form of mammogram imaging (FIG. 5A), the control unit 200 controls the radiation source moving unit 12, the detector moving unit 23, the elevating unit 24, and the housing elevating unit 53 to control the rotation and translation of the radiation tube 11, the rotation, translation, and movement (vertical movement) in the vertical direction of the radiation detector 21, and the movement (vertical movement) in the vertical direction of the housing support unit 41 (radiation imaging unit 2) so as to form the imaging geometric system shown in FIG. 5B.

When switching the imaging form, the control unit 200 controls the housing elevating unit 53 based on the imaging type to move (move downward) the housing support unit 41 by the distance D (=SODmam−SODct) based on the difference between SODmam in mammogram imaging and SODct in CBCT imaging, thereby controlling the position (vertical moving position) in the vertical direction of the radiation imaging unit 2 connected to the housing support unit 41.

Note that the switching of the imaging form is not limited to the switching from mammogram imaging to CT imaging (CBCT imaging), and the control of the control unit 200 is similarly applicable to a case in which switching from CT imaging (CBCT imaging) to mammogram imaging is done. That is, when mammogram imaging is selected from the form of CT imaging (CBCT imaging) (FIG. 5B), the control unit 200 can control the radiation source moving unit 12, the detector moving unit 23, the elevating unit 24, and the housing elevating unit 53 to control the rotation and translation of the radiation tube 11, the rotation and translation of the radiation detector 21, and the movement of the radiation detector 21 in the vertical direction so as to form the imaging geometric system shown in FIG. 5A.

When switching the imaging form, the control unit 200 controls the housing elevating unit 53 based on the imaging type to move (move upward) the housing support unit 41 by the distance D (=SODmam−SODct) based on the difference between SODmam in mammogram imaging and SODct in CBCT imaging, thereby controlling the position (vertical moving position) in the vertical direction of the radiation imaging unit 2 connected to the housing support unit 41.

The breast imaging apparatus includes the radiation imaging unit that holds the radiation generation unit and the radiation detection unit that detects radiation irradiation from the radiation generation unit such that they face each other, and the housing elevating unit capable of vertically moving the housing support unit that supports the radiation imaging unit. The breast imaging apparatus can perform mammogram imaging and CT imaging by the radiation imaging unit. A method of controlling a breast imaging apparatus includes a step (step S1) of controlling the vertical movement of the housing elevating unit by the control unit 200 based on an imaging type that is mammogram imaging or CT imaging. The breast imaging apparatus further includes the elevating unit 24 capable of vertically moving the detector of the radiation detection unit, and the selection unit 300 that selects the imaging type that is mammogram imaging or CT imaging. In the step (step S1) of the control method of the breast imaging apparatus, the control unit 200 controls the vertical movements of the housing elevating unit 53 and the elevating unit 24 based on the imaging type.

According to the arrangement of this embodiment, the position of the housing support unit that supports the radiation imaging unit can be controlled based on the imaging type. For example, even when performing mammogram imaging and CT imaging (CBCT imaging) for the same object or identical objects of different heights, the apparatus can be set in accordance with the position of the body part of the object to be imaged. This makes it possible to automatically set different imaging geometric systems in mammogram imaging and CT imaging and raise the throughput of imaging in both mammogram imaging and CT imaging.

### Second Embodiment

In the first embodiment, an arrangement in which one radiation tube 11 and one radiation detector 21 are moved for mammogram imaging and CBCT imaging has been described. In the second embodiment, an arrangement in which a radiation tube 11 and a radiation detector 21 are arranged for each of mammogram imaging and CBCT imaging to obviate the necessity of components (a radiation source moving unit 12 and a detector moving unit 23) used to move the radiation tube 11 and the radiation detector 21 in the rotation axis direction of a rotary frame 6 will be described.

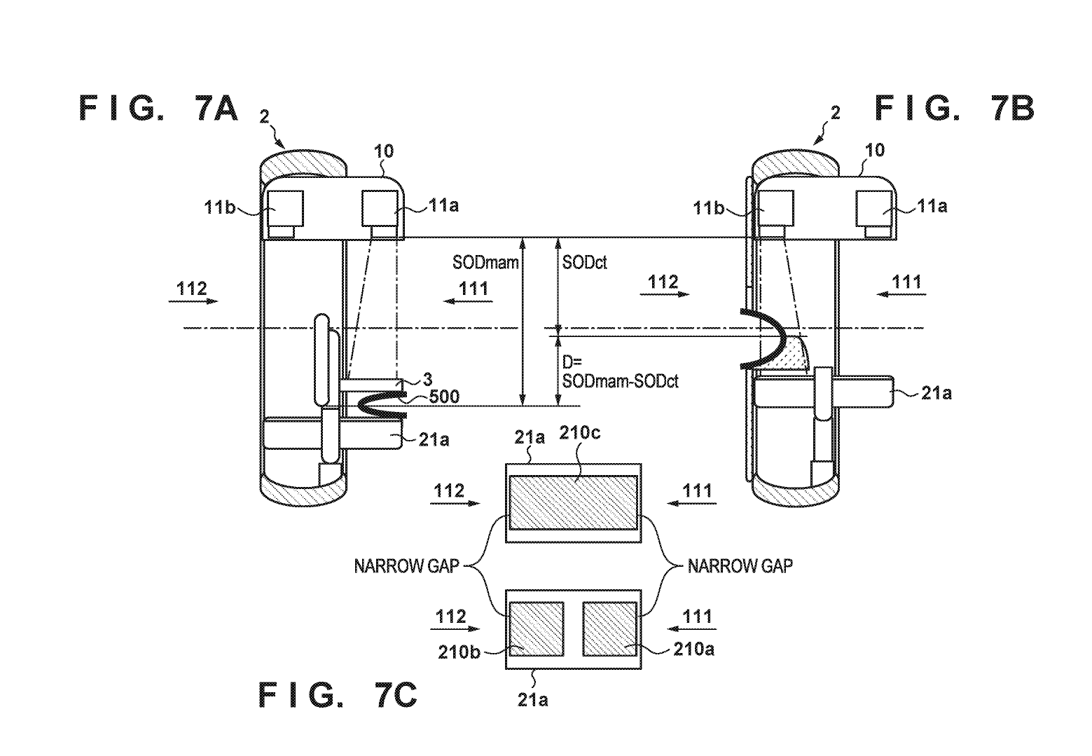

FIG. 7A is a view showing the state of a radiation imaging unit 2 at the time of mammogram imaging of a breast imaging apparatus 100 according to the second embodiment. FIG. 7B is a view showing the state of the radiation imaging unit 2 at the time of CBCT imaging of the breast imaging apparatus 100 according to the second embodiment. The radiation generation unit 10 includes a first radiation source for mammogram imaging and a second radiation source for CT imaging. That is, the radiation generation unit 10 includes a radiation tube lla for mammogram imaging and a radiation tube llb for CBCT imaging. A radiation detector 21a having a large detection area corresponds to both mammogram imaging from a first side 111 and CBCT imaging from a second side 112. As shown in FIG. 7C, the radiation detector 21a can be configured to have a large detection area 210c. Alternatively, the radiation detector 21a may be configured to have a first detector for mammogram imaging and a second detector for CT imaging. More specifically, the radiation detector 21a includes two detection areas 210a and 210b, and forms narrow gaps on both the first side 111 and the second side 112. Note that a grid 22 has a grid structure suitable for mammogram imaging and CBCT imaging in each of the detection areas 210a and 210b. Note that in place of the radiation detector 21a of a large area, two radiation detectors 21 as used in the first embodiment may be arranged. In this case, a control unit 200 can control the rotation of an elevating unit 24 to change the directions of the radiation detectors 21 in accordance with an imaging type. If the radiation detector 21a of a large area is used for imaging, the operation of changing the direction of the radiation detector in accordance with the imaging type is unnecessary. Hence, the control unit 200 controls the vertical movement of the elevating unit 24 to control the position of the radiation detector.

According to the second embodiment, the components (the radiation source moving unit 12 and the detector moving unit 23) used to move the radiation tube and the radiation detector in the rotation axis direction of the rotary frame 6 are unnecessary. Note that two radiation tubes may be arranged, as shown in FIGS. 7A and 7B, and the radiation detector 21 may be moved by the detector moving unit 23. Alternatively, two radiation detectors 21a of a large area or two radiation detectors 21 may be arranged, and the radiation tube 11 may be moved by the radiation source moving unit 12.

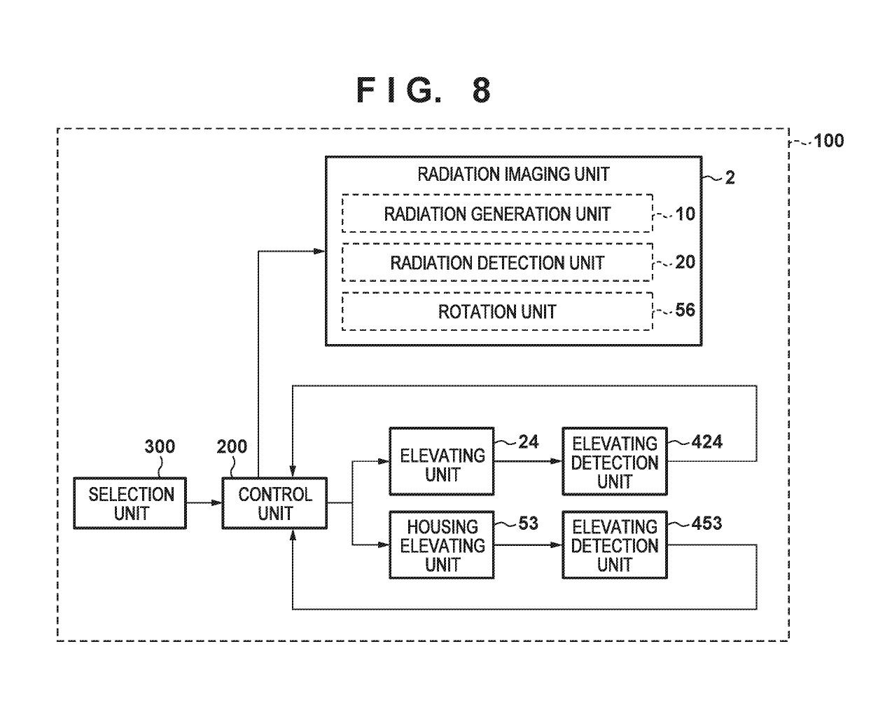

FIG. 8 is a block diagram for explaining processing of the control unit 200 of the breast imaging apparatus 100. FIG. 8 is different from the block diagram (FIG. 6) of the first embodiment in that the radiation source moving unit 12, the detector moving unit 23, detection units (a rotation detection unit 414 and a position detection unit 412) corresponding to the radiation source moving unit 12, and a position detection unit 423 corresponding to the detector moving unit 23 are not included in the block diagram.

In the breast imaging apparatus 100 according to the second embodiment, when mammogram imaging is selected, the control unit 200 controls an elevating unit 24 and a housing elevating unit 53 to control the position of the radiation detector 21 in the vertical direction and the position of a housing support unit 41 (radiation imaging unit 2) in the vertical direction so as to form the imaging geometric system shown in FIG. 7A.

When CT imaging (CBCT imaging) is selected from the form of mammogram imaging (FIG. 7A), the control unit 200 controls the elevating unit 24 and the housing elevating unit 53 to control the position of the radiation detector 21 in the vertical direction and the position of the housing support unit 41 (radiation imaging unit 2) in the vertical direction so as to form the imaging geometric system shown in FIG. 7B. When switching the imaging form, the control unit 200 controls the housing elevating unit 53 based on the imaging type to move (move downward) the housing support unit 41 by a distance D based on the difference between SODmam in mammogram imaging and SODct in CBCT imaging, thereby controlling the position (vertical moving position) in the vertical direction of the radiation imaging unit 2 connected to the housing support unit 41.

When mammogram imaging is selected from the form of CT imaging (CBCT imaging) (FIG. 7B), the control unit 200 controls the elevating unit 24 and the housing elevating unit 53 to control the position of the radiation detector 21 in the vertical direction and the position of the housing support unit 41 (radiation imaging unit 2) in the vertical direction so as to form the imaging geometric system shown in FIG. 7A. When switching the imaging form, the control unit 200 controls the housing elevating unit 53 based on the imaging type to move (move upward) the housing support unit 41 by the distance based on the difference between SODmam in mammogram imaging and SODct in CBCT imaging, thereby controlling the position (vertical moving position) in the vertical direction of the radiation imaging unit 2 connected to the housing support unit 41.

According to the arrangement of this embodiment, the position of the housing support unit that supports the radiation imaging unit can be controlled based on the imaging type, as in the first embodiment. This makes it possible to automatically set different imaging geometric systems in mammogram imaging and CT imaging and raise the throughput of imaging in both mammogram imaging and CT imaging. In addition, since the time for the rotation and translation of the radiation source and the translation of the radiation detector is unnecessary, it is possible to further improve the throughput of imaging and reduce the burden on the object at the time of imaging.

### 0ther Embodiments

Embodiment(s) of the present invention can also be realized by a computer of a system or apparatus that reads out and executes computer executable instructions (e.g., one or more programs) recorded on a storage medium (which may also be referred to more fully as a ‘non-transitory computer-readable storage medium’) to perform the functions of one or more of the above-described embodiment(s) and/or that includes one or more circuits (e.g., application specific integrated circuit (ASIC)) for performing the functions of one or more of the above-described embodiment(s), and by a method performed by the computer of the system or apparatus by, for example, reading out and executing the computer executable instructions from the storage medium to perform the functions of one or more of the above-described embodiment(s) and/or controlling the one or more circuits to perform the functions of one or more of the above-described embodiment(s). The computer may comprise one or more processors (e.g., central processing unit (CPU), micro processing unit (MPU)) and may include a network of separate computers or separate processors to read out and execute the computer executable instructions. The computer executable instructions may be provided to the computer, for example, from a network or the storage medium. The storage medium may include, for example, one or more of a hard disk, a random-access memory (RAM), a read only memory (ROM), a storage of distributed computing systems, an optical disk (such as a compact disc (CD), digital versatile disc (DVD), or Blu-ray Disc (BD)™, a flash memory device, a memory card, and the like.

While the present invention has been described with reference to exemplary embodiments, it is to be understood that the invention is not limited to the disclosed exemplary embodiments. The scope of the following claims is to be accorded the broadest interpretation so as to encompass all such modifications and equivalent structures and functions.

This application claims the benefit of Japanese Patent Application No. 2015-131840, filed Jun. 30, 2015, which is hereby incorporated by reference herein in its entirety.

What is claimed is:

1. A breast imaging apparatus capable of performing mammogram imaging and CT imaging by a radiation imaging unit that holds a radiation generation unit and a radiation detection unit configured to detect radiation irradiation from the radiation generation unit such that the radiation generation unit and the radiation detection unit face each other, comprising:
a housing elevating unit configured to vertically move a housing support unit configured to support the radiation imaging unit; and
a control unit configured to control the vertical movement of the housing elevating unit based on an imaging type that is one of mammogram imaging and CT imaging.

2. The apparatus according to claim 1, further comprising:
an elevating unit configured to vertically move a detector of the radiation detection unit; and
a selection unit configured to select the imaging type that is one of the mammogram imaging and the CT imaging,
wherein the control unit controls the vertical movements of the housing elevating unit and the elevating unit based on the imaging type.

3. The apparatus according to claim 2, wherein the control unit moves the elevating unit based on the imaging type so as to change a distance between the radiation generation unit and the radiation detection unit.

4. The apparatus according to claim 1, wherein the control unit moves the housing elevating unit based on the imaging type so as to change a position of the housing support unit in a vertical direction.

5. The apparatus according to claim 1, wherein when changing the imaging type between the mammogram imaging and the CT imaging, the control unit
obtains a distance based on a difference between a first distance between the radiation generation unit and a body part of an object to be imaged in the mammogram imaging and a second distance between the radiation generation unit and the body part of the object to be imaged in the CT imaging, and
moves the housing elevating unit based on the obtained distance.

6. The apparatus according to claim 5, wherein when changing the imaging type from the mammogram imaging to the CT imaging, the control unit moves the housing support unit downward based on the distance.

7. The apparatus according to claim 5, wherein when changing the imaging type from the CT imaging to the mammogram imaging, the control unit moves the housing support unit upward based on the distance.

8. The apparatus according to claim 5, wherein the control unit moves the housing support unit such that the distance based on the difference becomes zero.

9. The apparatus according to claim 2, wherein the radiation imaging unit further comprises:
a ring-shaped rotary frame in which the radiation generation unit and the radiation detection unit are fixed such that the radiation generation unit and the radiation detection unit face each other; and
a fixed frame configured to rotatably hold the rotary frame.

10. The apparatus according to claim 1, wherein the radiation generation unit includes a first radiation source for the mammogram imaging and a second radiation source for the CT imaging.

11. The apparatus according to claim 1, wherein the radiation detection unit includes a first detector for the mammogram imaging and a second detector for the CT imaging.

12. The apparatus according to claim 9, wherein the radiation generation unit includes a radiation source moving unit configured to move a radiation source in a direction along a rotation axis of the rotary frame,
the radiation detection unit includes a detector moving unit configured to move the detector in the direction along the rotation axis of the rotary frame, and
the control unit controls the movements of the radiation source moving unit and the detector moving unit based on the imaging type.

13. The apparatus according to claim 12, wherein the radiation source moving unit is configured to rotate the radiation source about a rotation axis in a direction intersecting the rotation axis of the rotary frame, and
the control unit controls the rotation of the radiation source by the radiation source moving unit based on the imaging type.

14. The apparatus according to claim 12, wherein the elevating unit is configured to rotate the detector of the radiation detection unit about a rotation axis in a direction intersecting the rotation axis of the rotary frame, and
the control unit changes a direction of the detector of the radiation detection unit by controlling the rotation of the elevating unit based on the imaging type.

15. The apparatus according to claim 14, wherein the detector of the radiation detection unit has a narrow gap indicating that a width of one side of a detection area is narrower than widths of remaining sides, and
the control unit controls the rotation of the elevating unit based on the imaging type such that the narrow gap of the detector is directed to an object.

16. The apparatus according to claim 2, wherein the control unit controls the elevating unit and the housing elevating unit based on the selected imaging type, and provides different imaging geometric systems in the mammogram imaging and the CT imaging.

17. A method of controlling a breast imaging apparatus including a radiation imaging unit that holds a radiation generation unit and a radiation detection unit configured to detect radiation irradiation from the radiation generation unit such that the radiation generation unit and the radiation detection unit face each other, and a housing elevating unit configured to vertically move a housing support unit configured to support the radiation imaging unit, and capable of performing mammogram imaging and CT imaging by the radiation imaging unit, comprising:
controlling, by a control unit, the vertical movement of the housing elevating unit based on an imaging type that is one of mammogram imaging and CT imaging.

18. A computer-readable storage medium storing a program that causes a computer to execute each step of a method of controlling a breast imaging apparatus including a radiation imaging unit that holds a radiation generation unit and a radiation detection unit configured to detect radiation irradiation from the radiation generation unit such that the radiation generation unit and the radiation detection unit face each other, and a housing elevating unit configured to vertically move a housing support unit configured to support the radiation imaging unit, and capable of performing mammogram imaging and CT imaging by the radiation imaging unit, the method of controlling the breast imaging apparatus comprising:
controlling, by a control unit, the vertical movement of the housing elevating unit based on an imaging type that is one of mammogram imaging and CT imaging.

19. A breast imaging apparatus capable of performing mammogram imaging and CT imaging by a radiation imaging unit that holds a radiation generation unit and a radiation detection unit configured to detect radiation irradiation from the radiation generation unit such that the radiation generation unit and the radiation detection unit face each other, comprising:
a housing elevating unit configured to vertically move a housing support unit configured to support the radiation imaging unit; and
a control unit configured to control the vertical movement of the housing elevating unit.
